(19) United States
(12) Patent Application Publication
(10) Pub. No.: US 2016/0178519 A1
(43) Pub. Date: Jun. 23, 2016

(54) Marker For Detection And Confirmation Of Peripheral Lung Nodules

(71) Applicant: BOSTON SCIENTIFIC SCIMED, INC., Maple Grove, MN (US)

(72) Inventors: Michael E. Zupkofska, Rockland, MA (US); Mahfuza Ahmed, BROOKLINE, MA (US); Michael M. Borek, Leomonster, MA (US); Gerald Fredrickson, Westford, MA (US); Paul Smith, Smithfield, RI (US); Douglas C. Shepard, Mansfield, MA (US); Michael D. Sinisi, Boston, MA (US)

(21) Appl. No.: 14/948,727

(22) Filed: Nov. 23, 2015

Related U.S. Application Data
(60) Provisional application No. 62/096,222, filed on Dec. 23, 2014.

(51) Int. Cl. G01N 21/64; A61M 15/00; G01N 23/22; G01N 27/72; A61M 5/00; A61M 5/178

(52) U.S. Cl. CPC G01N 21/6428; A61M 5/007; A61M 5/178; G01N 23/22; G01N 27/72; A61M 15/009; A61B 2010/045

(57) ABSTRACT

An example method for obtaining a biopsy sample may include delivering a tumor marker to a patient, guiding a biopsy tool to a desired biopsy region within a patient's body, obtaining a biopsy sample from the desired biopsy region, removing the biopsy sample from the patient's body, and after removing the biopsy sample from the patient's body, scanning the biopsy sample to detect the presence of the tumor marker in the patient.

### PRIORITY CLAIM

The present disclosure claims priority to U.S. Provisional Patent Application Ser. No. 62/096,222 filed Dec. 23, 2014; the disclosure of which is incorporated herewith by reference.

### TECHNICAL FIELD

The present disclosure pertains to medical devices, and methods for manufacturing and/or using medical devices. More particularly, the present disclosure pertains to obtaining a biopsy sample and confirming the sample was obtained from the targeted region.

### BACKGROUND

A wide variety of medical devices have been developed for medical use, for example, pulmonary use. Some of these devices include catheters, stents, diagnostic tools, and the like, and delivery devices and/or systems used for delivering such devices. These devices are manufactured by any one of a variety of different manufacturing methods and may be used according to any one of a variety of methods. Of the known medical devices, delivery system, and methods, each has certain advantages and disadvantages. There is an ongoing need to provide alternative medical devices and delivery devices as well as alternative methods for manufacturing and using medical devices and delivery devices.

### BRIEF SUMMARY

This disclosure provides design, material, manufacturing methods, and use alternatives for medical devices, including biopsy devices and methods. An example method and kit for performing a biopsy is disclosed. An example method for performing a biopsy may comprise:

guiding a biopsy tool to a desired biopsy region within a patient's body, the desired biopsy region including a tissue previously marked with a tumor marker;

obtaining a biopsy sample from the desired biopsy region;

removing the biopsy sample from the patient's body; and

after removing the biopsy sample from the patient's body, scanning the biopsy sample to detect the presence of the tumor marker in the patient;

wherein if the biopsy sample is positive for the tumor marker the biopsy sample has been obtained from the desired biopsy region and if the biopsy sample is negative for the tumor marker the biopsy sample has not been obtained from the desired biopsy region.

Alternatively or additionally to any of the embodiments above, wherein if the sample is negative for the tumor marker, the method further comprising the steps of guiding a biopsy tool to the desired biopsy region within a patient's body, obtaining a biopsy sample from the desired biopsy region, removing the biopsy sample from the patient's body, and after removing the biopsy sample from the patient's body, scanning the biopsy sample to detect the presence of the tumor marker in the patient are repeated during a same medical procedure until a biopsy sample is positive for the tumor marker.

Alternatively or additionally to any of the embodiments above, wherein the tumor marker comprises methylene blue.

Alternatively or additionally to any of the embodiments above, wherein the tumor marker comprises gold nanoparticles.

Alternatively or additionally to any of the embodiments above, wherein the tumor marker comprises quantum dots.

Alternatively or additionally to any of the embodiments above, wherein the tumor marker is selected from the group of paramagnetic nanoparticles, Fc protein coated nanoparticles, or a biodegradable nanoparticles.

Alternatively or additionally to any of the embodiments above, wherein the tumor marker was previously injected into the patient.

Alternatively or additionally to any of the embodiments above, wherein the tumor marker was previously inhaled by the patient.

Alternatively or additionally to any of the embodiments above, wherein delivering the tumor marker comprises delivering an ingestible tumor marker to the patient.

Alternatively or additionally to any of the embodiments above, wherein scanning the biopsy sample comprises viewing the sample using at least one of a laser scanning confocal microscope, a fluorescence microscope, a white light microscope, or a near infrared light or a hand held source of illumination.

Alternatively or additionally to any of the embodiments above, wherein scanning the biopsy sample comprises viewing the sample using at least one of a Raman spectroscopy, optical induced fluorescence, x-ray radiation or a Hall-effect sensor.

Alternatively or additionally to any of the embodiments above, wherein the tumor marker comprises a combination of methylene blue, gold nanoparticles, quantum dots, paramagnetic nanoparticles, Fc protein coated nanoparticles, and/or a biodegradable nanoparticle.

An example kit for performing a biopsy may comprise:

a catheter having a proximal end region, a distal end region, and a lumen extending between the proximal end region and the distal end region;

a biopsy tool;

a syringe having a tubular cavity, a plunger, and a needle; and

a vial, the vial containing a marking agent that accumulates preferentially in tumorous tissues.

Alternatively or additionally to any of the embodiments above, wherein the marking agent is selected from the group of methylene blue, gold nanoparticles, quantum dots, paramagnetic nanoparticles, Fc protein coated nanoparticles, or a biodegradable nanoparticles.

An example kit for performing a biopsy may comprise:

a catheter having a proximal end region, a distal end region, and a lumen extending between the proximal end region and the distal end region;

a biopsy tool;

an inhaler; and

a canister, the canister containing a marking agent that accumulate preferentially in tumorous tissues.

Alternatively or additionally to any of the embodiments above, wherein the marking agent is selected from the group of methylene blue, gold nanoparticles, quantum dots, paramagnetic nanoparticles, Fc protein coated nanoparticle, or a biodegradable nanoparticle.

An example method for performing a biopsy, the method may comprise:

guiding a biopsy tool to a desired biopsy region within a patient's body, the desired biopsy region including a tissue previously marked with a tumor marker;

obtaining a biopsy sample from the desired biopsy region;

removing the biopsy sample from the patient's body; and

after removing the biopsy sample from the patient's body, scanning the biopsy sample to detect the presence of the tumor marker in the patient;

wherein if the biopsy sample is positive for the tumor marker the biopsy sample has been obtained from the desired biopsy region and if the biopsy sample is negative for the tumor marker the biopsy sample has not been obtained from the desired biopsy region.

Alternatively or additionally to any of the embodiments above, wherein if the sample is negative for the tumor marker, the method further comprising the steps of guiding a biopsy tool to the desired biopsy region within a patient's body, obtaining a biopsy sample from the desired biopsy region, removing the biopsy sample from the patient's body, and after removing the biopsy sample from the patient's body, scanning the biopsy sample to detect the presence of the tumor marker in the patient are repeated during a same medical procedure until a biopsy sample is positive for the tumor marker.

Alternatively or additionally to any of the embodiments above, wherein a waiting period allows the tumor marker to permeate a tumor tissue and/or allows non-absorbed marker to clear from adjacent tissues.

Alternatively or additionally to any of the embodiments above, wherein the tumor marker comprises methylene blue.

Alternatively or additionally to any of the embodiments above, wherein the tumor marker comprises gold nanoparticles.

Alternatively or additionally to any of the embodiments above, wherein the tumor marker comprises quantum dots.

Alternatively or additionally to any of the embodiments above, wherein the tumor marker comprises paramagnetic nanoparticles.

Alternatively or additionally to any of the embodiments above, wherein the tumor marker comprises biodegradable nanoparticles or Fc protein coated nanoparticles.

Alternatively or additionally to any of the embodiments above, wherein the tumor marker comprises a combination of methylene blue, gold nanoparticles, quantum dots, paramagnetic nanoparticles, Fe protein coated nanoparticles, and/or a biodegradable nanoparticle.

Alternatively or additionally to any of the embodiments above, wherein the tumor marker was previously injected into the patient.

Alternatively or additionally to any of the embodiments above, wherein the tumor marker was previously inhaled by the patient.

Alternatively or additionally to any of the embodiments above, wherein the tumor marker was previously ingested by the patient.

Alternatively or additionally to any of the embodiments above, wherein scanning the biopsy sample comprises viewing the sample using at least one of a laser scanning confocal microscope, a fluorescence microscope, a white light microscope, a near infrared light or a hand held source of illumination.

Alternatively or additionally to any of the embodiments above, wherein scanning the biopsy sample comprises viewing the sample using at least one of a Raman spectroscopy, optical induced fluorescence, x-ray radiation, or a Hall-effect sensor.

An example kit for performing a biopsy may comprise:

a catheter having a proximal end region, a distal end region, and a lumen extending between the proximal end region and the distal end region;

a biopsy tool;

a syringe having a tubular cavity, a plunger, and a needle; and

a vial, the vial containing a marking agent that accumulates preferentially in tumorous tissues.

Alternatively or additionally to any of the embodiments above, wherein the marking agent is selected from the group of methylene blue, gold nanoparticles, quantum dots, paramagnetic nanoparticles, Fc protein coated nanoparticles, or biodegradable nanoparticles.

Alternatively or additionally to any of the embodiments above, wherein the biopsy tool comprises a biopsy needle.

An example kit for performing a biopsy may comprise:

a catheter having a proximal end region, a distal end region, and a lumen extending between the proximal end region and the distal end region;

a biopsy tool;

an inhaler; and

a canister, the canister containing a marking agent that accumulate preferentially in tumorous tissues.

Alternatively or additionally to any of the embodiments above, wherein the biopsy tool comprises a biopsy needle.

Alternatively or additionally to any of the embodiments above, wherein the marking agent is selected from the group of methylene blue, gold nanoparticles, quantum dots, paramagnetic nanoparticles, Fe protein coated nanoparticles, or biodegradable nanoparticles.

Alternatively or additionally to any of the embodiments above, wherein the marking agent is stored with a propellant.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a plan view of an example biopsy tool accessing a peripheral lung nodule;

FIG. 2 is a flow chart of an illustrative biopsy procedure;

FIG. 3 is a partial perspective view of an illustrative nodule on a portion of the lung;

FIG. 4 is a partial perspective view of an illustrative biopsy tool retrieving a biopsy from an illustrative nodule;

FIG. 5 is a plan view of an illustrative biopsy sample on a slide;

FIG. 6 is a plan view of the illustrative biopsy sample of FIG. 5 under illumination;

FIG. 7 is a plan view of another illustrative biopsy sample under illumination;

FIG. 8 is an illustrative kit for marking and obtaining a biopsy sample; and

FIG. 9 is another illustrative kit for marking and obtaining a biopsy sample.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

The global lung cancer epidemic, combined with the adoption of lung cancer screening, may result in an increasing number of suspicious solitary pulmonary nodules (SPNs) found on chest computed tomography (CT) scans. Suspicious SPNs, which typically exist in the periphery of the lungs, may be difficult to access and diagnose using current bronchoscopic technologies designed primarily for the central airway. Peripheral lung nodules, or solitary pulmonary nodules (SPNs), may be rounded masses measuring up to 3 centimeters (cm), which can be benign or malignant. When a SPN is identified, it may need to be diagnosed with a biopsy. In some instances, once a biopsy sample has been obtained, the sample may be sent to a lab where it is analyzed using histology. Until the results from the histology are returned, the physician performing the biopsy may not know if the biopsy sample was obtained from the targeted region. It may be desirable to provide a device and/or system to confirm a collected sample came from the targeted lesion in real time during the biopsy procedure. This may allow a physician to obtain additional biopsy samples as needed during the same procedure if it is determined that the original sample was not obtained from the targeted lesion. While the present disclosure is described with respect to lung nodules, it is contemplated that the methods and devices described herein can be applied to other parts of the anatomy, such as, but not limited to gastrointestinal, urological, gynecological, etc.

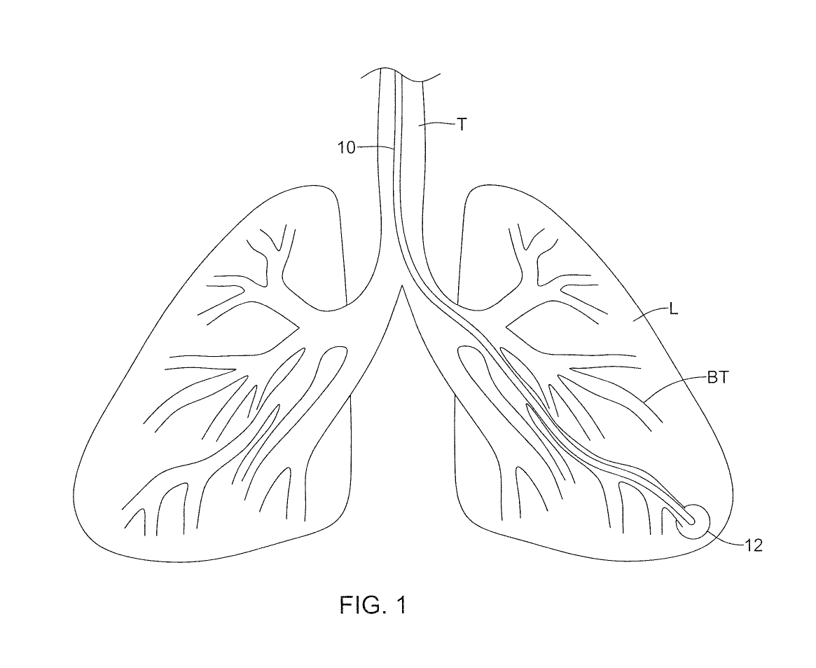

FIG. 1 illustrates a plan view of an example biopsy system 10 advanced through the trachea T and the bronchial tree BT to a peripheral nodule 12 within the lung L. In some instances, the nodule or lesion 12 may be located in a peripheral region of the lung which may be difficult to access and visualize. It may be desirable to aid in the visualization and confirmation of cancerous and/or benign nodules located in the lungs. In some instances, when administered into the body, some molecules, and/or other engineered particles, such as but not limited to, certain nanoparticles, may accumulate in tumorous tissue much more than normal tissue. The underlying mechanism for their entrapment is known as the Enhanced Permeability and Retention (EPR) effect. This biological phenomenon may be attributed to the rapid and uncontrolled growth of tumors, resulting in leaky vasculature within a cancerous mass. The EPR effect may allow a marking agent, such as but not limited to certain molecules and/or engineered particles, to accumulate in a lesion. Nanoparticles, or markers, can be utilized in the early detection of peripheral lung nodules or those which are visible on a CT scan. The markers can be introduced into the body via inhalation, ingestion, injection, or a combination thereof, prior to a visit with the patient's physician or at the beginning of a visit. Once within the bloodstream, the markers are able to travel throughout the body. Those that enter a tumor and become trapped in the tissue will remain immobilized while those that remain in the bloodstream will eventually be cleared from the body. In conjunction with a catheter, these markers can then be used as a guide for the surgeon to improve the accuracy of biopsied tissue samples during navigation and following retrieval of the sample. It is contemplated that the presence or absence of the marker in a biopsy sample may be used to determine if the sample was obtained from the targeted lesion, as will be discussed in more detail below.

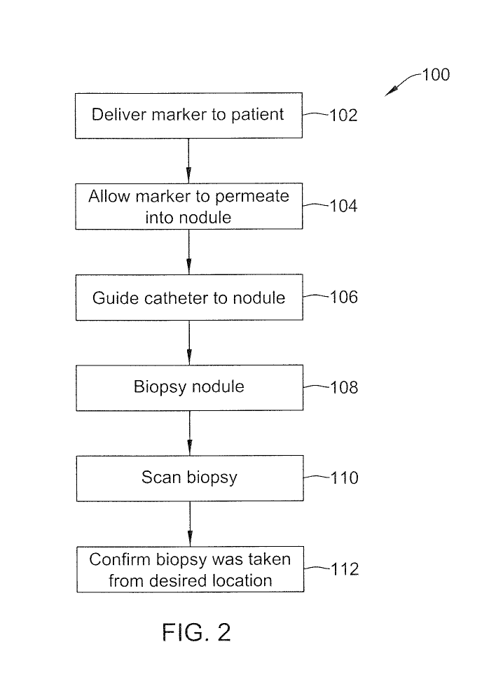

FIG. 2 illustrates a flow chart of a brief overview of an illustrative biopsy procedure 100 for obtaining a biopsy and confirming the biopsy was obtained from the nodule or desired biopsy region. To begin the procedure 100, a marking agent or tumor marker may be delivered or administered to a patient, as shown in step 102. In some instances, the marker may delivered locally near the desired biopsy region. In other instances, the marker may be delivered systemically. Once the marker has been delivered to the patient, in the next step 104 of the procedure the marker may be allowed to permeate into the nodule. In some instances, a time period in the range of 20-30 minutes, in the range of several hours, in the range of a day, in the range of several days, or more may be necessary to allow the marker to permeate into the nodule. It is contemplated that the time period may be selected to not only allow the marker to permeate into the nodule, but also allow the non-absorbed marker to clear from the healthy tissue. It is contemplated that in some instances, the marker may not need to be 100% clear from healthy tissues for the healthy tissue to be sufficiently clear of the marker. In some instances, if the marker is not allowed to clear from the healthy tissues, some, most, or all of the samples may test positive for cancerous or benign tumors, even when the sample was not taken from a cancerous or benign tumor. Once the marker has permeated the nodule 12, the patient may be prepped for the surgical portion of the biopsy procedure 100. In some instances, the patient may be prepped for the surgical portion of the biopsy before step 102 or during step 104. The next step 106 in the biopsy procedure may be to guide a biopsy tool to the desired biopsy region. In some instances, a catheter may be advanced through a bronchoscope. The catheter may be guided to the nodule. In the next step 108, a biopsy needle, or other biopsy device, may be used to obtain a sample of the nodule. The sample may then be removed from the body. The biopsy sample may be scanned in the procedure room, or nearby facility, using, for example, a near infrared light source, to determine if the biopsy sample was taken from the desired biopsy region, as shown in step 110. The marker that has permeated into the nodule may fluoresce or otherwise illuminate when exposed to the necessary wavelength of light which may allow a physician to confirm the sample was taken from the desired location, as shown in step 112. For example, if the sample fluoresces under the light source, the physician can confirm the biopsy was indeed taken from the suspect nodule or desired biopsy region. If the sample does not fluoresce under the light source, the biopsy sample was likely not obtained from the suspect nodule or desired biopsy region. If the biopsy sample was not taken from the suspect nodule or desired biopsy region, the physician may repeat steps 106, 108, 110, and 112 of the biopsy procedure 100 until the physician confirms the sample was taken from the suspect nodule or desired biopsy region. This may allow the physician to confirm the biopsy sample was taken from the biopsy region.

As noted above, peripheral lung nodules may be difficult to access and to visualize. The illustrative biopsy procedure 100 may help to alleviate the struggle of identifying the location and confirmed collection of cancerous or suspect tissue. Without the ability to confirm in real time the biopsy sample was taken from the suspect nodule or biopsy region, the physician may not know if the sample was taken from the biopsy region until the histology report is returned. In the event the sample was not taken from the biopsy region, the patient may need to undergo another sedation and surgical procedure in an attempt to obtain a biopsy from the suspect nodule. Confirming the sample was taken from the biopsy region in real time, or while the patient is still prepped for the biopsy procedure may reduce the need for future procedures in the event the sample was not obtained from the biopsy region.

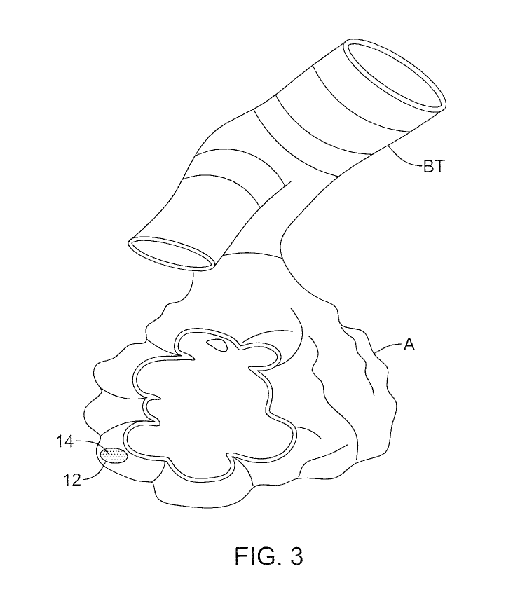

The illustrative biopsy procedure 100 will now be described in more detail with respect to FIGS. 3-7. In some instances, the marker may be injected into the patient using a syringe. In other instances, the marker delivered in an inhalable form. For example, the marker may be delivered using an inhaler or a respiratory mask. It is contemplated that other known drug delivery techniques may also be used. For example, in some instances, the marker may be ingested or absorbed through the skin. In some instances, nanoparticles or markers can be utilized in the early detection of peripheral lung nodules or those which are visible on a CT scan. These can be introduced into the body prior to a visit with the patient's physician or at the beginning of the visit. Once within the bloodstream, the markers are able to travel throughout the body. Those that enter a tumor and become trapped in the tissue will remain immobilized. Referring to FIG. 3, which illustrates a nodule 12 on the alveoli A of the lungs, the marker 14 may preferentially accumulate in a nodule or tumor 12. In some instances, the nodule may be a solitary pulmonary nodule (SPN) located in the periphery of the lungs. Those that remain in the bloodstream will eventually be cleared by the body's renal system. In some instances, the markers 14 can also be used as a guide for the physician to improve the accuracy of biopsied tissue samples during navigation and following retrieval of the sample.

It is contemplated that the marker 14 may be a material or particle that accumulates preferentially in tumorous tissues, such as, but not limited to methylene blue, gold nanoparticles, quantum dots (silicon), or paramagnetic nanoparticles. These are just examples. It is contemplated that the size of the particles forming marking 14 may be in the range of approximately 10-300 nanometers (nm). However, the particle size may be smaller than 10 nm or larger than 300 nm as desired. The type of marker 14 used may be selected for each particular procedure or biopsy. Methylene blue may be absorbed and retained by both benign and malignant lesions. Gold may have limited interaction with the body's immune system due to its inert nature. Gold may also be visible on an x-ray or fluoroscopic real-time image, therefore making it possible to see the nodule while navigating to it helping to guide the physician to the nodule 12. In addition, a fluorescent marker or chromophore can be added to the surface of the gold nanoparticle to make its presence easily identifiable (when exposed to the necessary wavelength of light) after retrieval of a tissue sample. Similar to gold, paramagnetic materials (such as, for example, iron oxide) may also be visible during fluoroscopy. Their magnetic attraction could be used to track the particle's location, and therefore, the lesion's location. In conjunction with a catheter that has a probe/sensor to generate and detect the strength of a magnetic field, the paramagnetic particles may behave as a beacon to target the lesion real-time based on factors unaffected by visual limitation. The particle size of the paramagnetic materials may be in the range of approximately 10-50 nm. Quantum dots can be manufactured through existing technology and used in conjunction with bronchoscopic catheter tissue sampling. Quantum dots have specific bright fluorescent properties which would facilitate detection through use of fluorescence microscopy examination (or equivalent optical techniques) of the suspect tissue samples. In some embodiments, the marker 14 may be an Fc protein coated nanoparticle. It is contemplated that the Fc protein coated nanoparticle may be ingested and absorbed through the intestinal wall. In some instances, the marker may be biodegradable, a biodegradable nanoparticle with or without a fluorescent, or may include a biodegradable coating applied to the marker. It is contemplated this may better control the duration of the markers' presence in the body. In some embodiments, a combination of two or more different markers 14 may be used to enhance the effects of the tumor marker. For example, the marker 14 may be a combination of methylene blue, gold nanoparticles, quantum dots (silicon), paramagnetic nanoparticles, an Fc protein coated nanoparticle, and/or a biodegradable nanoparticle with or without a fluorescent.

Once the marker 14 has been delivered to the patient in step 102, the marker 14 may be allowed to permeate into the nodule 12 as shown in step 104 of FIG. 2. In some instances, a time period in the range of 20-30 minutes, in the range of several hours, in the range of a day, in the range of several days, or more may be necessary to allow the marker to permeate into the nodule. Once the marker 14 has permeated the nodule 12, the patient may be prepped for a biopsy procedure. In some instances, the patient may be prepped for the surgical portion of the biopsy before delivering the marker or during while the marker is allowed to permeate into the nodule. It is further contemplated that the marker may be delivered to the patient after prepping the patient for the biopsy procedure.

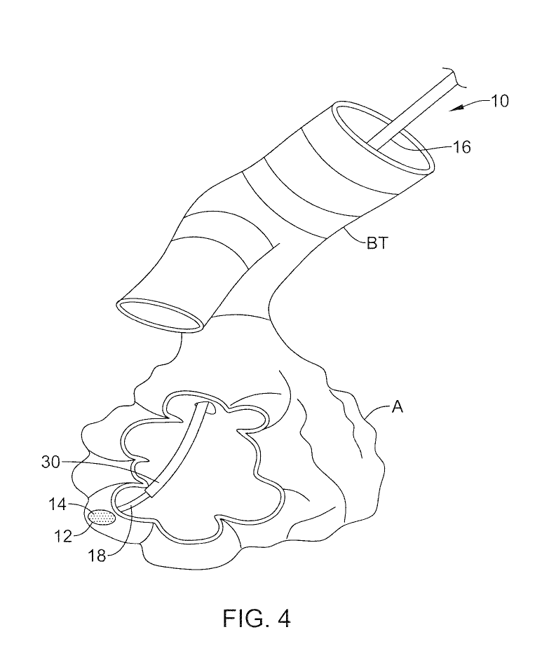

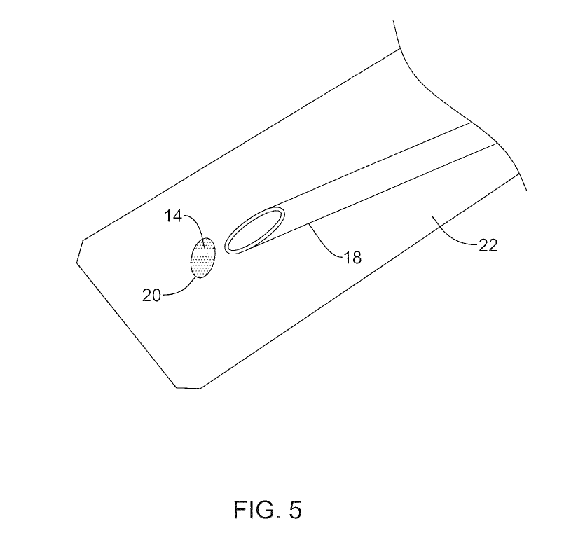

Referring additionally to FIG. 4, a biopsy system 10 may be advanced through the trachea T and the bronchial tree BT towards the nodule 12 as indicated in step 106 of FIG. 2. In some embodiments, the biopsy system 10 may include a bronchoscope (not explicitly shown), a catheter 16, and/or a biopsy needle 18. The biopsy system 10, or components, thereof may be provided along with a device for delivering the marker and the marker as a kit, although this is not required. In some instances, the catheter 16 may be steerable to facilitate guiding the distal end 30 to the biopsy region or nodule 12. It is contemplated that the catheter 16 may be advanced through a working lumen of a bronchoscope or other guide device. In some instances, the marker 14 may be used to track the location of the marker 14 and/or nodule 12. The distal end 30 of the catheter 16 may be provided with a fiber optic probe (not explicitly shown) to visually confirm the presence of the marker 14 in the nodule 12 in real time. It is contemplated that the probe may transmit and detect signals to measure reflectance based on the specific wavelength of radiant light associated with the marker 14 deployed. In some instances, the distal end 30 of the catheter may be provided with a probe and/or sensor to generate and detect the strength of a magnetic field. When paramagnetic markers are used, the marker 14 may behave as a beacon to target the nodule 12 using properties unaffected by visual limitations. The biopsy needle 18, or other biopsy device, may be advanced through a working lumen of the steerable catheter 16. The biopsy tool 18 may be used to obtain a biopsy sample 20 (shown in FIG. 5) from the nodule 12 or targeted lesion, as indicated in step 108 of FIG. 2.

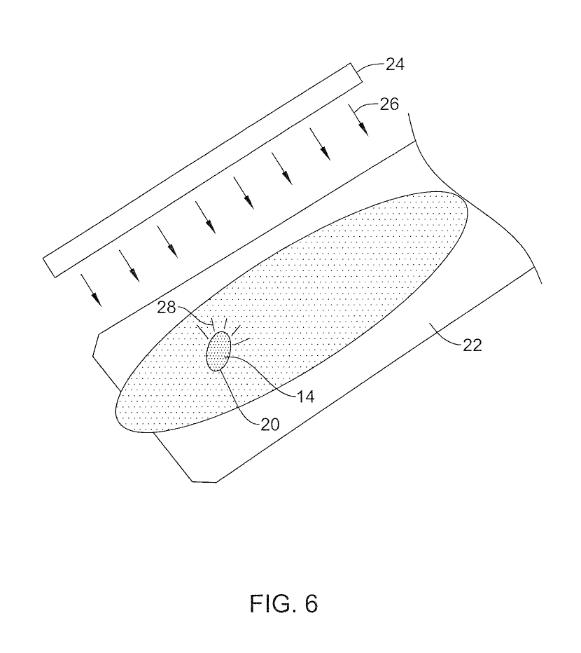

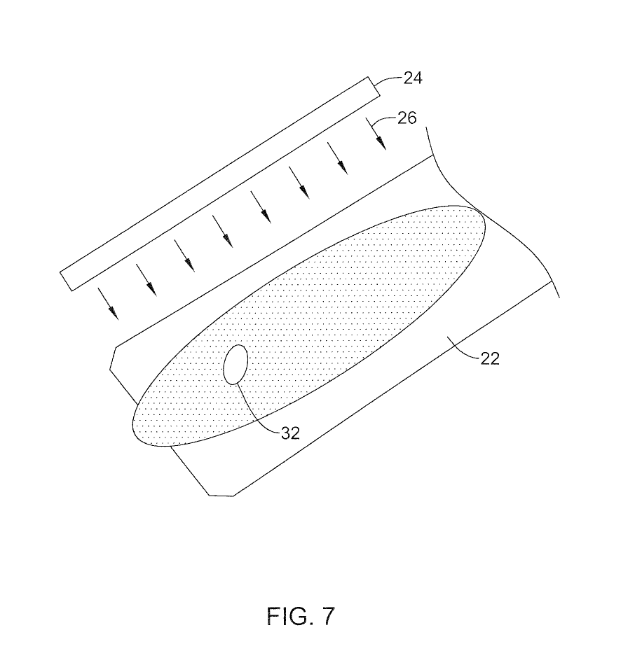

Once the biopsy sample 20 has been removed from the patient's body, the biopsy sample 20 may be placed on a slide 22 or otherwise prepared for analysis, as shown in FIG. 5. Referring additionally to FIG. 6, the biopsy sample 20 may then be scanned with an appropriate medium to determine if the markers are present in the sample, as indicated in step 110 of FIG. 2. For example, the sample 20 may be illuminated with a light 24, or other scanning system, having the appropriate wavelength 26 for the marker 14 used. Suitable scanning systems may include, but are not limited to, microscopy by white light, near-infrared (NIR) fluorescence, Raman spectroscopy, optical (for example, laser) induced fluorescence, Hall-effect sensor, x-ray radiation, etc. In some embodiments, the scanning systems may be handheld illumination sources. In some instances, a near-infrared fluorescence imaging system may be used to illuminate the sample 20. In other instances, a laser scanning confocal microscope or fluorescence microscopy may be used to view or scan the sample 20. It is contemplated that the light or scanning system 24 may be selected based on the specific wavelength of radiant light associated with the marker 14 deployed. If the biopsy sample 20 was taken from the nodule 12 including markers 14, the sample 20 may fluoresce 28 or otherwise have a visually recognizable feature, such as a glow or emit a color. FIG. 7 illustrates another illustrative biopsy sample 32 on a slide for analysis. The biopsy sample 30 does not include any markers. Therefore, when the sample 32 is illuminated 26 by an appropriate medium 24, the sample 32 will not have any visual change. For example, the sample 32 will not fluoresce. In the event the sample 32 was not taken from the biopsy region, the physician may retrieve another biopsy sample while the patient is still prepped for the biopsy procedure. Confirming the sample was taken from the biopsy region in real time, or while the patient is still prepped for the biopsy procedure may reduce the need for future procedures in the event the sample was not obtained from the biopsy region.

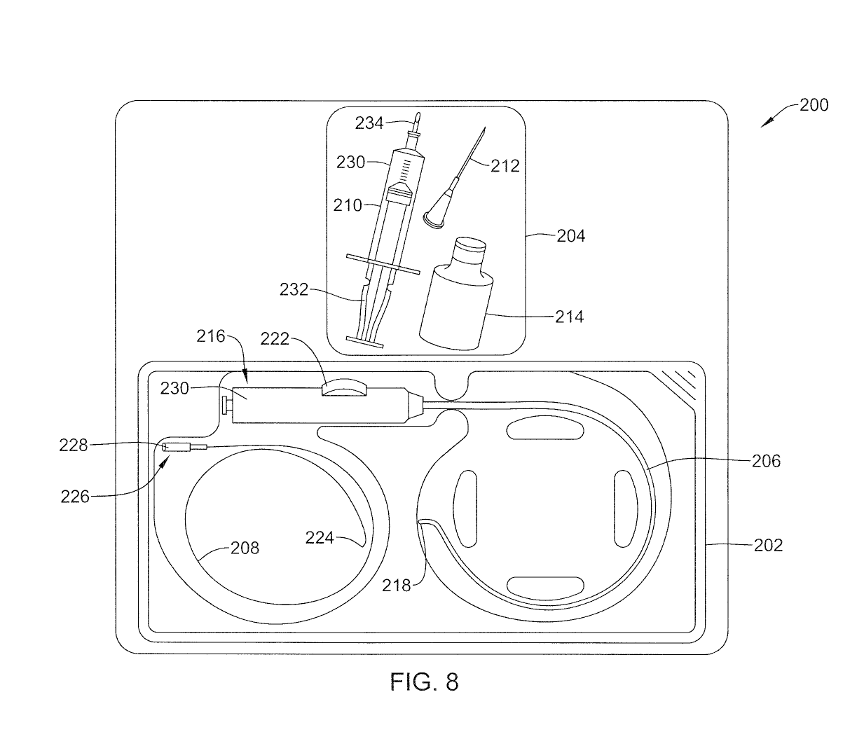

FIG. 8 illustrates an exemplary kit 200 that may be used to perform the illustrative biopsy procedure 100 described above. The kit 200 may include a first portion 202 including one or more devices to access the nodule or biopsy region and a second portion 204 including devices for delivering the marker and the marker itself. The first portion 202 may include a catheter 206 and a biopsy tool 208. In some instances, the distal end region 218 of the catheter 206 may include a fiber optic probe or a probe and/or sensor to generate and detect the strength of a magnetic field, although this is not required. The catheter 206 may have a long, elongated, flexible tubular configuration that may be inserted into a patient's body for a medical diagnosis/treatment. The catheter 206 may extend proximally from a distal end region 218 to a proximal end region 216. The proximal end 216 of the catheter 206 may include a hub or handle 220 attached thereto for connecting other treatment devices or providing a port for facilitating other treatments. In some instances, the handle 220 may include an actuator 222 for manipulation of a steering mechanism within the catheter 206. It is contemplated that the stiffness of the catheter 206 may be modified for use in various lumen diameters and various locations within the body. The catheter 206 may include one or more lumens extending between the proximal end region 216 and the distal end region 218. In some embodiments, the biopsy tool 208 may be a biopsy needle. However, other biopsy devices can be provided. A biopsy needle 208 may include a sharp, hollow distal end 224 to pierce and retain a body tissue. The proximal end 226 of the biopsy needle 208 may include a handle or gripping portion 228.

The second portion 204 of the kit 200 may include a syringe 210 for delivering or injecting a marker into the patient's body. The syringe 210 may include a tubular cavity 230, a plunger 232, and a needle 234. The plunger 232 may be slidably disposed within the tubular cavity 230. In some instances, a second alternative needle 212 may also be provided. The second portion 204 may further include a vial or container 214 containing a marker, nanoparticle or marking agent that accumulates preferentially in tumorous tissues, such as marker 14 described above. For example, the vial 214 may include methylene blue, gold nanoparticles, quantum dots, and/or paramagnetic nanoparticles. The marker may be a liquid or dissolved in a biocompatible liquid for injection into the body.

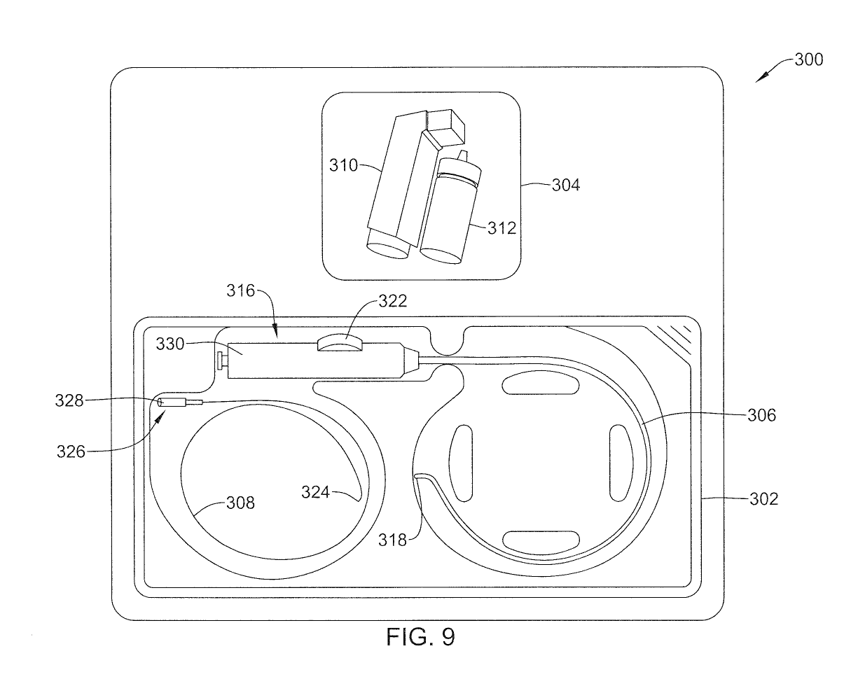

FIG. 9 illustrates another exemplary kit 300 that may be used to perform the illustrative biopsy procedure 100 described above. The kit 300 may include a first portion 302 including devices to access the nodule and a second portion 304 including devices for delivering the marker and the marker itself. The first portion 302 may include a catheter 306 and a biopsy tool 308. In some instances, the distal end region 318 of the catheter 306 may include a fiber optic probe or a probe and/or sensor to generate and detect the strength of a magnetic field, although this is not required. The catheter 306 may have a long, elongated, flexible tubular configuration that may be inserted into a patient's body for a medical diagnosis/treatment. The catheter 206 may extend proximally from a distal end region 318 to a proximal end region 316. The proximal end 316 of the catheter 306 may include a hub or handle 320 attached thereto for connecting other treatment devices or providing a port for facilitating other treatments. In some instances, the handle 320 may include an actuator 322 for manipulation of a steering mechanism within the catheter 306. It is contemplated that the stiffness of the catheter 306 may be modified for use in various lumen diameters and various locations within the body. The catheter 306 may include one or more lumens extending between the proximal end region 316 and the distal end region 318. In some embodiments, the biopsy tool 308 may be a biopsy needle. However, other biopsy devices can be provided. A biopsy needle 308 may include a sharp, hollow distal end 324 to pierce and retain a body tissue. The proximal end 326 of the biopsy needle 308 may include a handle or gripping portion 328. The second portion 304 of the kit 300 may include an inhaler 310 for delivering a marker, nanoparticle or marking agent that accumulates preferentially in tumorous tissues, such as marker 14 described above, into the patient's body. The marker may be provided in a pressurized canister 312. For example, the canister 312 may include methylene blue, gold nanoparticles, quantum dots, and/or paramagnetic nanoparticles. In some instances, the marker may be stored in solution with a propellant within the canister 312. In other instances, the marker may be stored as a suspension. The inhaler 310 and canister 312 may be used to deliver the marker directly into the lungs. For example, the canister 312 may be engaged with the inhaler to deliver an aerosolized marker directly into the lungs.

The materials that can be used for the various components of the biopsy devices, systems, kits, or components thereof, such as devices 10/100/200 (and/or other structures disclosed herein) and the various members disclosed herein may include those commonly associated with medical devices. For simplicity purposes, the following discussion makes reference the devices 10/100/200 and components of thereof. However, this is not intended to limit the devices and methods described herein, as the discussion may be applied to other similar systems and/or components of systems or devices disclosed herein.

The devices 10/100/200 and/or other components of delivery system may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the polymer can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: 30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the devices 10/100/200 and/or other components of delivery system may be doped with, made of, or otherwise include a radiopaque material. Radiopaque materials are understood to be materials capable of producing a relatively bright image on a fluoroscopy screen or another imaging technique during a medical procedure. This relatively bright image aids the user of the devices 10/100/200 in determining its location.

Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the devices 10/100/200 to achieve the same result.

In some embodiments, a degree of Magnetic Resonance Imaging (MRI) compatibility is imparted into the devices 10/100/200. For example, devices 10/100/200, or portions or components thereof, may be made of a material that does not substantially distort the image and create substantial artifacts (i.e., gaps in the image). Certain ferromagnetic materials, for example, may not be suitable because they may create artifacts in an MRI image. The devices 10/100/200, or portions thereof, may also include and/or be made from a material that the MRI machine can image. Some materials that exhibit these characteristics include, for example, tungsten, cobalt-chromium-molybdenum alloys (e.g., UNS: R30003 such as ELGILOY, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1-15. (canceled)

16. A method for performing a biopsy, the method comprising:
guiding a biopsy tool to a desired biopsy region within a patient's body, the desired biopsy region including a tissue previously marked with a tumor marker;
obtaining a biopsy sample from the desired biopsy region;
removing the biopsy sample from the patient's body; and
after removing the biopsy sample from the patient's body, scanning the biopsy sample to detect the presence of the tumor marker in the patient;
wherein if the biopsy sample is positive for the tumor marker the biopsy sample has been obtained from the desired biopsy region and if the biopsy sample is negative for the tumor marker the biopsy sample has not been obtained from the desired biopsy region.

17. The method of claim 16, wherein if the sample is negative for the tumor marker, the method further comprising the steps of guiding a biopsy tool to the desired biopsy region within a patient's body, obtaining a biopsy sample from the desired biopsy region, removing the biopsy sample from the patient's body, and after removing the biopsy sample from the patient's body, scanning the biopsy sample to detect the presence of the tumor marker in the patient are repeated during a same medical procedure until a biopsy sample is positive for the tumor marker.

18. The method of claims 16, wherein a waiting period allows the tumor marker to permeate a tumor tissue and/or allows non-absorbed marker to clear from adjacent tissues.

19. The method of claim 16, wherein the tumor marker comprises methylene blue.

20. The method of claim 16, wherein the tumor marker comprises gold nanoparticles.

21. The method of claim 16, wherein the tumor marker comprises quantum dots.

22. The method of claim 16, wherein the tumor marker comprises paramagnetic nanoparticles.

23. The method of claim 16, wherein the tumor marker comprises biodegradable nanoparticles or Fc protein coated nanoparticles.

24. The method of claim 16, wherein the tumor marker comprises a combination of methylene blue, gold nanoparticles, quantum dots, paramagnetic nanoparticles, Fc protein coated nanoparticles, and/or a biodegradable nanoparticle.

25. The method claim 16, wherein the tumor marker was previously injected into the patient.

26. The method of claim 16, wherein the tumor marker was previously inhaled by the patient.

27. The method of claim 16, wherein scanning the biopsy sample comprises viewing the sample using at least one of a laser scanning confocal microscope, a fluorescence microscope, a white light microscope, a near infrared light, or a hand held source of illumination.

28. The method of claim 16, wherein scanning the biopsy sample comprises viewing the sample using at least one of a Raman spectroscopy, optical induced fluorescence, x-ray radiation, or a Hall-effect sensor.

29. A kit for performing a biopsy, the kit comprising:
a catheter having a proximal end region, a distal end region, and a lumen extending between the proximal end region and the distal end region;
a biopsy tool;
a syringe having a tubular cavity, a plunger, and a needle; and
a vial, the vial containing a marking agent that accumulates preferentially in tumorous tissues.

30. The kit of claim 29, wherein the marking agent is selected from the group of methylene blue, gold nanoparticles, quantum dots, paramagnetic nanoparticles, Fc protein coated nanoparticles, or biodegradable nanoparticles.

31. The kit of claim 29, wherein the biopsy tool comprises a biopsy needle.

32. A kit for performing a biopsy, the kit comprising:
a catheter having a proximal end region, a distal end region, and a lumen extending between the proximal end region and the distal end region;
a biopsy tool;
an inhaler; and
a canister, the canister containing a marking agent that accumulate preferentially in tumorous tissues.

33. The kit of claim 32, wherein the biopsy tool comprises a biopsy needle.

34. The kit of claim 32, wherein the marking agent is selected from the group of methylene blue, gold nanoparticles, quantum dots, paramagnetic nanoparticles, Fc protein coated nanoparticles, or biodegradable nanoparticles.

35. The kit of claim 32, wherein the marking agent is stored with a propellant.
